(19) United States
(12) Patent Application Publication
(10) Pub. No.: US 2011/0009938 A1
(43) Pub. Date: Jan. 13, 2011

(54) IMPLANTABLE MEDICAL LEAD AND METHOD FOR MANUFACTURE THEREOF

(75) Inventor: Kenneth Dowling, Bro (SE)

Correspondence Address: SCHIFF HARDIN, LLP;PATENT DEPARTMENT, 233 S. Wacker Drive-Suite 6600, CHICAGO, IL, 60606-6473, US

(73) Assignee: ST JUDE MEDICAL AB, Jarfalla (SE)

(21) Appl. No.: 12/922,279

(22) PCT Filed: May 8, 2008
(86) PCT No.: PCT/SE08/00313

§ 371 Date: Sep. 13, 2010

(30) Foreign Application Priority Data
Apr. 29, 2008 (SE) 2008/000299

(51) Int. Cl. A61N 1/00; B32B 5/14

(52) U.S. Cl. 607/119; 607/116; 264/171.13

(57) ABSTRACT

An implantable medical lead for implantation in a patient which has at least one electrical conductor connected to at least one electrode and/or sensor of said lead. The at least one conductor is arranged within a continuous sheet of a polymer material. A distal portion of the lead is adapted to be located in or at a heart of said patient and a proximal portion of said lead is connectable to an implantable medical device and arranged such that, when connected to the device, at least a part of the proximal portion of the sheet is placed in close proximity to said medical device. At least the proximal portion of the polymer sheet material is processed in at least a first heat process stage such that an inherent resistance to wear of the polymer sheet material is substantially maintained, and the distal portion of said polymer sheet material is processed in at least a second heat process stage in which a polymer morphology of said polymer material is altered such that an inherent flexibility of the polymer sheet material is substantially increased.

### BACKGROUND OF THE INVENTION

1. Field of the Invention

The present invention generally relates to the field of implantable medical devices. More specifically, the present invention relates to an implantable medical lead for implantation in a patient, the lead having a distal end adapted to be located within or at a heart of the patient and a proximal end connectable to an implantable medical device.

2. Description of the Prior Art

Within the field of implantable medical device, such as heart stimulators or pacemakers, implantable leads are used for conveying electrical stimuli from the device to a distal portion of the lead, e.g. to the myocardium of a human heart, for instance the endocardium and to transfer signals, for example, signal representative of electrical activity of the heart to the device. The requirements of cardiac leads with respect to material properties or characteristics are contradictory which may be difficult to combine. For example, a distal portion of the cardiac lead needs to be flexible yet having a satisfactory degree of stiffness such that the portion easily can adapt to and follow the curvature of the implantation path within the vessels and to avoid perforation or tearing of vessel walls and tissue walls, such as a heart wall. Conversely, the remaining portion and in particular the proximal portion or end is relatively stiff or rigid to ease insertion of the lead during implantation procedure, i.e. a manipulation of the proximal end allows the distal end to be operated. Further, the proximal portion which is connected to the device should also have a wear resistible surface to cope with the wear due to friction between the proximal portion and the can of the device. Thus, because of the compromise between the different material properties required by the material used in a cardiac lead, this is a complex issue.

As indicated above, a problem is abrasion or wear of the portion of the lead that is in contact with the medical device when implanted. More specifically, during an implantation procedure extra or additional lead wire is provided at the site where the medical device is implanted. This is a security measure for the purpose of compensating body movements which otherwise could cause stretching of the lead. Since the medical device, which conventionally is implanted in a subcutaneous pocket, is also more or less fixated, such stretching could, in absence of excess lead wire, cause a stressed distal end. As a result, the portion of the distal end, including e.g. a helix, that is secured to a target tissue, e.g. a heart wall, could cause damages to the tissue. Thus, a coil of excess lead wire is conventionally implanted together with medical device to avoid that situation, which achieves a resilient effect between the two fixation points of the distal end and proximal end of the lead.

However, when implanted the medical lead abuts against the surface of the medical device since they are located close to each other and friction between the can of the device and the lead portion abutting the can due to, for example, body movements may cause wear on the lead surface. Thereby, the lead surface which is subjected to wear or abrasion needs to be provided with an inherent resistance against such wearing and tearing.

In practice, a material is often selected as a compromise between wear resistant and flexibility properties, being optimized for neither.

The combined effects of these problems result in an implantable medical lead having a distal end portion which is flexible and a proximal portion sufficiently rigid to maneuver the distal end and wherein at least the proximal end portion is abrasion resistant. In the prior art, the medical lead may be constructed by individual portions, a distal portion and a proximal portion which are assembled by means of a intermediate joint or seem. These individual parts or components may be made of different material and individually treated to obtain a desirable mechanical property suitable for its purpose. However, such a solution only solves part of the complex problem, but more important this solution increases significantly the complexity of joining these components and the difficulty of assembly and manufacturing thereof.

One way of addressing part of the problem presented above is described in U.S. Pat. No. 5,171,383. Here a catheter guide wire is disclosed wherein a core member is made of an elastic alloy. This elastic alloy is subjected to a heat treatment process along its longitudinal direction such that the rigidity of the proximal end portion becomes comparatively high and the flexibility of the distal end is increased. This differential heat treatment provides a catheter guide wire having a flexible distal end to avoid buckling deformations and tissue wall perforations, and a rigid proximal end to achieve a good torque transmitting performance to the distal end portion. However, this solution does not solve the problem of wear of the surface of the lead.

In U.S. Pat. No. 4,963,306, another technique is disclosed which presents a method for making a fuseless soft tip angiographic catheter. A fuseless polymeric tube having a body portion and a tip portion is provided, wherein the body portion is heat treated while the tip portion is maintained at a temperature lower than the heat treatment temperature. Thereby, a polymeric tube having a tip portion and a body portion with different physical properties. Thus, the soft tip is flexible such that the catheter may is able to reach distant vessels without damaging or tearing the lining of the blood vessels. However, this is a catheter device for an insertion procedure and not intended for implantation. Also, this prior art does not address the problem related to the wear of the surface of the lead.

Consequently, there is a need within the art of implantable medical leads that enables a durable and reliable implantable medical lead in combination with an accurate and easy implantation procedure thereof.

### SUMMARY OF THE INVENTION

Thus, an object of the present invention is to provide an improved implantable medical lead which alleviates the problem mentioned above.

Another object of the present invention is to provide an improved medical lead having a prolonged life time in comparison with prior art medical lead.

A further object of the present invention is to provide an improved method for selectively designing the properties of a material for use in a medical lead.

These and other objects are achieved by providing an implantable medical lead, a method for manufacturing thereof and use of an implantable polymer material or Elast-Eon 2A®-material in an implantable medical lead.

According to a first aspect of the present invention, there is provided an implantable medical lead for implantation in a patient comprising at least one electrical conductor connected to at least one electrode and/or sensor of said lead, said at least one conductor being arranged within a continuous sheet of a polymer material, wherein a distal portion of said lead is adapted to be located in or at a heart of the patient and wherein a proximal portion of the lead is connectable to an implantable medical device and arranged such that, when connected to the device, at least a part of the proximal portion is placed in close proximity to the medical device, wherein the proximal portion of the polymer sheet material is processed in at least a first heat process stage such that an inherent resistance to wear of the polymer sheet material is substantially maintained; and said distal portion of the polymer sheet material is processed in at least a second heat process stage in which a polymer morphology of the polymer material is altered such that an inherent flexibility of the polymer sheet material is substantially increased.

A second aspect of the present invention provides a method for manufacturing of an implantable polymer sheet material for implantation in a patient, wherein a distal portion of the polymer sheet material is adapted to be located in or at a heart of the patient and wherein a proximal portion of the polymer sheet material is connectable to an implantable medical device and arranged such that, when connected to the device, at least a part of the proximal portion is placed in close proximity to the medical device. The method includes the steps of providing a continuous sheet of a polymer material; processing at least the proximal portion of the polymer sheet material in at least a first heat process stage such that an inherent resistance to wear of the polymer sheet material is substantially maintained; processing the distal portion in at least a second heat process stage in which a polymer morphology of the polymer material is altered such that an inherent flexibility of the polymer sheet material is substantially increased.

A third aspect of the present invention provides a method for manufacturing of an implantable medical lead for implantation in a patient, wherein a distal portion of the lead is adapted to be located in or at a heart of the patient and wherein a proximal portion of the lead is connectable to an implantable medical device and arranged such that, when connected to the device, at least a part of the proximal is placed in close proximity to the medical device. The method includes the steps of providing at least one electrical conductor connected to at least one electrode and/or sensor of the lead; providing a continuous sheet of a polymer material; processing at least the proximal portion of the polymer sheet material in at least a first heat process stage such that an inherent resistance to wear of the polymer sheet material is substantially maintained; processing the distal portion in at least a second heat process stage in which a polymer morphology of the polymer material is altered such that an inherent flexibility of the polymer sheet material is substantially increased; and assembling the at least one conductor with the polymer sheet material.

The polymer sheet may be Elast-Eon 2A®)-material according to the invention.

Thus, the present invention is based on the insight of using a single or continuous implantable polymer sheet material that can be heat processed to obtain selectable material properties at different parts of a processed material piece for achieving an implantable medical lead that is capable of uniting the contradicting requirements put upon such leads, i.e. a medical lead having a distal end portion which is flexible and a proximal portion sufficiently rigid to maneuver the distal end and wherein at least the proximal end portion is abrasion resistant. The implantable polymer sheet material functions is heat treated at individual portions to obtain an end portion having an high flexibility and wherein at least a proximal end portion is heat treated such that a high abrasion or wear resistance can be achieved. This achieves an improved medical lead having one end for placement within or at a heart having a high degree of flexibility, and at least a portion of the opposite end, i.e. a proximal end, having a high resistance to wear or abrasion. Moreover, the use of a continuous sheet material and the fact that different parts of the sheet can be selectively processed eliminates unwanted joints or seems that could cause cracks or breakage. Thus, this enhances the reliability and durability even further.

The polymer sheet material used in the medical lead according to the first aspect enables an implantable medical lead having an enhanced abrasion or wear resistance at a selected portion, for example, the proximal end, which is in contact, or which at least frequently abuts, with medical device, and a flexible distal end which provides for a reliable and accurate connection between the lead and the heart. More specifically, a distal end, being secured to a portion of the heart tissue, which is flexible, enables a secure and reliable fixation point. Also, the flexible distal end portion facilitates an implantation or insertion of the lead.

In an embodiment of the first aspect of the invention, the polymer is a semi-crystalline copolymer having at least a soft amorphous segment and at least a hard crystalline segment being at least partially crystallized. It should be noted that the term “copolymer” as used herein is intended to refer to a polymer material that is derived from two (or more) monomers or monomeric species. Moreover, the term “semi-crystalline” as used herein is intended to refer to a polymer which is constituted by an amorphous and a crystalline region or section. A soft segment material is an elastomeric polymeric material that is amorphous and has a crystalline or glassy state that occurs at or above its intended use temperature, e.g. about 37° C. for implanted materials, and exhibits large degree of localized chain mobility. A hard segment is an elastomeric polymeric material that is crystalline or in an amorphous glassy state at and/or above the intended use temperature, and is characterized by a very low degree of localized chain mobility. The soft and hard regions or segments are phase separated meaning that the polymer material has elastomeric with elastomeric properties, such as elasticity, Thereby, the flexibility of the medical lead is enhanced which, in turn, facilitates and simplifies the insertion of the lead during the implantation procedure. Furthermore, during use, i.e. when the lead is implanted into a patient, the lead may easily follow the bodily movements.

In another embodiment of the first aspect of the invention, at least a portion of the amorphous segment has at least one flexible polymeric material from a group containing silicone, polyethers, polyethylene oxide, polyolefins, polycarbonates, or a combination thereof, and wherein at least a portion of the crystalline segment comprises at least one crystallizable polymeric material from a group containing aromatic urea, aromatic or aliphatic urethane. Such a material is often called a polyurethane material, a polyurea, or a polyurea-urethane. A preferred material comprises a linear block copolymer of hard and soft segments, and there are not chemical crosslinks between polymer chains to form a 3-D network, making these thermoplastics. At use temperature, crystallization between hard segments on the same or different chains give rise to a thermally-reversible network to give rise to the desired mechanical properties. Heating above the crystalline and glass transition temperatures gives rise to a melt which can be formed and processed as a thermoplastic according to known art. Hence, the preferred materials are classified as thermoplastic elastomers, of which thermoplastic urethanes is a preferred sub-group. A lead having such a polymer further enhances the elasticity of the polymer and thereby the flexibility of the lead.

In yet another embodiment of the first aspect of the invention, wherein the at least said proximal portion of the polymer sheet material is heat treated at a temperature interval from 50 to 100° C. during a period of about 30 minutes to about 5 hours and the distal portion of the polymer sheet material is heat treated at a temperature of at least 10° C. above the temperature of the first heat process stage during a period of at least about 5 minutes. Alternatively, the distal portion is heat treated at a temperature of about 120° C. during a period of about 30 minutes.

In yet another embodiment of the second or third aspect of the invention, the step of providing a continuous sheet of a polymer material is providing a semi-crystalline copolymer having at least a soft amorphous segment and at least a hard crystalline segment being at least partially crystallized.

In another embodiment of the second or third aspect of the invention, the step of providing a continuous sheet of a polymer material is providing a semi-crystalline copolymer having at least a soft amorphous segment where at least a portion thereof comprises at least one flexible polymeric material from a group containing silicone, polyethers, polyethylene oxide, polyolefins, polycarbonates, or a combination thereof, and having at least a hard crystalline segment where at least a portion thereof comprises at least one crystallizable polymeric material from a group containing aromatic urea, aromatic or aliphatic urethane.

Moreover, in another embodiment of the second or third aspect of the invention, the first heat process stage includes heating within a temperature interval from about 50 to about 100° C. during a period of about 30 minutes to about 5 hours, and wherein the second heat process stage comprises heating at a temperature at least about 10° C. above the temperature of the first heat process stage during a period of at least about 5 minutes. Alternatively, the second heat process stage comprises heating at a temperature of about 120° C. during a period of about 30 minutes.

In yet another embodiment of the second or third aspect of the invention, the first and second heat process stage take place simultaneously by means of a common oven that provides individual heat treatments to individual portions of the polymer sheet material. It is to be understood that such an oven may be arranged in various ways as long as a differentially heating is provided. For example, such an oven may be divided by oven walls such that the oven chamber is divided into at least to separate compartment, wherein each of these can provide individual heat treatments for the portion within the compartment.

### BRIEF DESCRIPTION OF THE DRAWINGS

The features that characterize the invention, both as to organization and to method of operation, together with further objects and advantages thereof, will be better understood from the following description used in conjunction with the accompanying drawings. It is to be expressly understood that the drawings is for the purpose of illustration and description and is not intended as a definition of the limits of the invention. These and other objects attained, and advantages offered, by the present invention will become more fully apparent as the description that now follows is read in conjunction with the accompanying drawings.

FIG. 1 illustrates the general principle of a medical lead in relation to a heart of a patient and a medical device.

FIG. 2 illustrates the relationship between abrasion resistance and hardness of a silicon elastomer material.

FIG. 3 illustrates the relationship between abrasion resistance and hardness of a polymer sheet material according to the invention.

FIG. 4 illustrates the relationship between stiffness as a function of heat treatment temperature of a polymer sheet material according to the invention.

FIG. 5 shows a block diagram illustrating the principles of a process according to the present invention.

### DESCRIPTION OF THE PREFERRED EMBODIMENTS

The following is a description of exemplifying embodiments in accordance with the present invention. This description is intended for describing the general principles of the invention and is not to be taken in a limiting sense. Please note that like reference numerals indicate structures or elements having same or similar functions or constructional features.

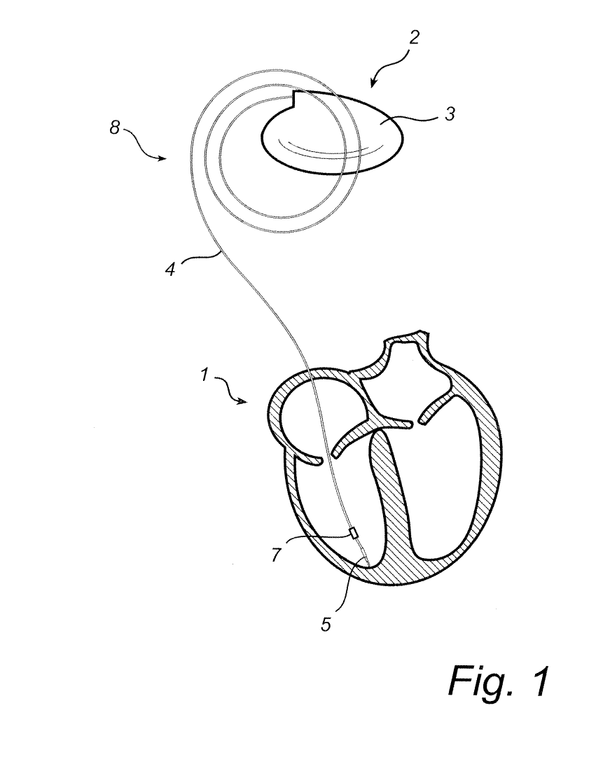

Referring first to FIG. 1, there is shown an implantable medical device or heart stimulator 2 in electrical communication with a human heart 1 via an implantable medical lead or cardiac lead 4 arranged for stimulation and sensing. Moreover, the heart stimulator 2 includes electronic circuitry and a battery contained within a hermetically sealed pacemaker housing 3. The housing 3 has a metallic casing of a biocompatible material, for example, titanium, enclosing the electronic circuitry and battery, and a molded plastic header portion, comprising connector blocks and apertures for receiving the connectors at the proximal ends of the cardiac leads. Also, at the proximal end of the lead, a coil of excess lead 8 is provided, which is to be implanted together with the medical device 2.

The electronic circuitry includes at least one pulse generator for generating stimulation pulses, sensing circuitry for receiving cardiac signals sensed by the cardiac lead 2, and a controller. The controller controls both the sensing of cardiac signals and the delivery of stimulation pulses, for instance as to the duration, energy content and timing of the stimulation pulses.

The stimulation pulses generated by the pulse generator are transmitted via the cardiac lead 4 and delivered to the cardiac tissue by the use of tip electrodes positioned at the distal end 5 of the cardiac lead. Generally, the tip electrode acts as the cathode when the cardiac pulse is delivered. Furthermore, in unipolar cardiac systems, the casing 3 acts as the anode, while in bipolar cardiac systems, the anode is provided by an annular or ring electrode 7 arranged on the cardiac lead at a small distance from the tip electrode.

It should be noted that even though a ring electrode 7 is illustrated in the greatly simplified drawing of FIG. 1, the present invention is equally applicable to unipolar, bipolar, and multipolar systems. Thus, implantable leads with or without ring electrodes are equally contemplated without departing from the scope of the invention. Furthermore, even though only one lead 4 for attachment and stimulation in the right ventricle is illustrated in the drawing, the medical implant 2 may be connected to further leads, for instance for stimulation of the right atrium, the left atrium, and/or the left ventricle.

The implantable medical lead 4 according to the present invention preferably includes at least one electrical conductor connected to at least one electrode and/or sensor of the lead, the at least one conductor being arranged within a continuous sheet of a polymer material. It should be noted that such a polymer sheet material may have the shape of a tube or the like provided with at least one lumen. In other words, the at least one electrical conductor is situated within a polymer sheet material, e.g. an isolating polymer tube

At least a portion of the proximal end of the lead has a maximized resistance to wear, or at least those parts which may be in contact with the device 2 when implanted. For example, that part of the lead that is implanted into the subcutaneous pocket is preferably subjected to a heat treatment such that the wear resistance of the polymer sheet material is substantially maintained. In FIG. 1, the lead 4 is provided with a wear or abrasion resistance surface property, which more or less equals a part of the lead 8 being located in the subcutaneous pocket and in proximity to the pocket, i.e. the proximal end portion. Preferably, the part of the lead that is less flexible, i.e. the proximal end, has a high resistance to wear. However, as is understood, the wear resistant property may also be arranged in other ways. For example, only the part of the lead being placed within the pocket may be processed such the inherent wear resistance property is maintained. Similarly, the distal end or a distal portion of the lead is subjected to heat treatment such that the inherent flexibility property of the polymer sheet material is increased. In other words, the distal end, or at least a portion thereof, is more flexible than the proximal end, or at least a portion thereof. In FIG. 1, about 10 cm is flexible (not shown) of the about 50 cm long lead. The distal treatment should be applied to at least about 5 cm of the distal end, and preferably about 10 to about 25 cm, which is the distal portion of the lead that is situated within the heart.

### Experiment 1

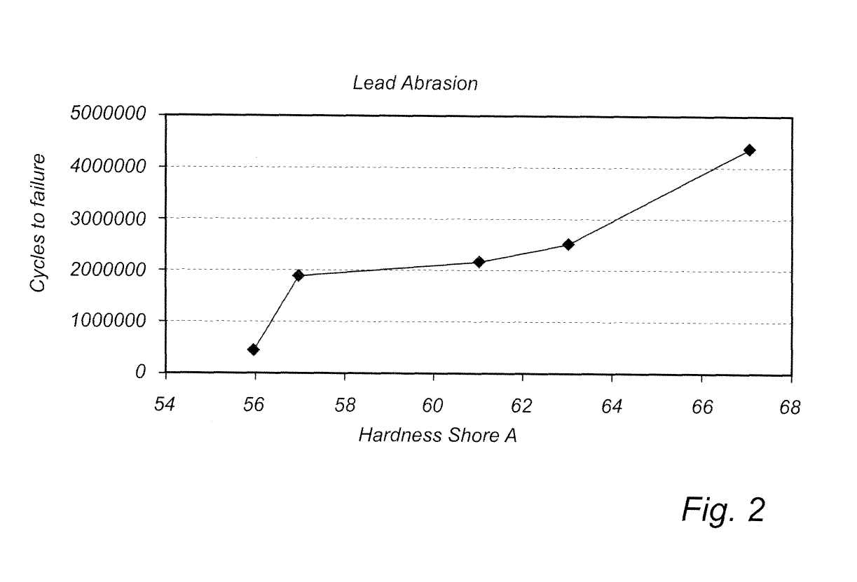

FIG. 2 presents experimental information showing that the hardness property and abrasion resistance property are related. The test result which relates to a silicone elastomer shows that within a group of otherwise chemically identical, the abrasion resistance increases with increasing hardness. The silicone elastomer or rubbers were cured and post-cured to achieve a specific hardness (Shore A). These were then tested in an abrasion test apparatus, which by St. Jude Medical internally is designated ES-1907 rev X1, designed for measuring the abrasion resistance of pacemaker lead bodies. For this type of abrasion, it has thus been demonstrated that harder materials, of otherwise identical composition as that of the present invention, have greater resistance to abrasion. Thus, it is beneficial to provide softness in the tip for flexibility, but retain hardness in the proximal end to optimize abrasion resistance.

### Experiment 2

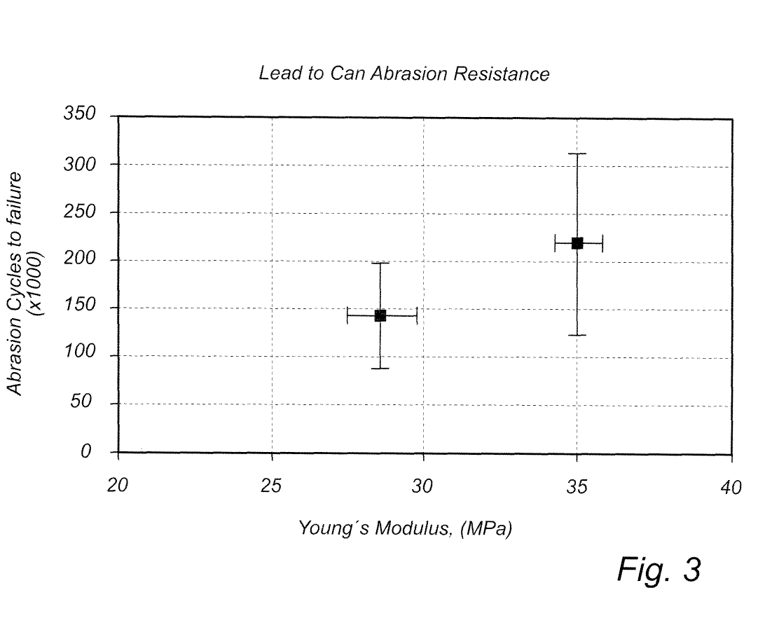

In FIG. 3, test results for a material according to an embodiment of the present invention, similar to that of the experiment 1, is shown. The graph in FIG. 3 presents abrasion resistance results on a pacemaker lead body made of an Elast-Eon material, more specifically an Elast-Eon 2A material. This material is provided by Aor-Tech. The purpose of such an experiment is to simulate the wear situation of a medical lead in abutment with a medical device can when implanted. The experiment was similarly performed as experiment 1 in the way that the material was first subjected to heat treatment followed by an abrasion test. The abrasion test was performed according to a St. Jude Medical internal test method called 60010764 rev P02. The result shows that the lower hardness material, i.e. less stiff as indicated by lower Young's modulus, from a heat treatment at 120 C/6 hrs has a lower abrasion resistance compare to material treated at 85 C/4 h that has higher stiffness/hardness/modulus, and thus higher abrasion resistance.

According to an embodiment of the present invention, the at least proximal portion of the polymer sheet material has a hardness ranging from Shore 60 A to Shore 80 D. Thus, the at least proximal portion of the medical lead is provided with an inherent wear resistance property.

### Experiment 3

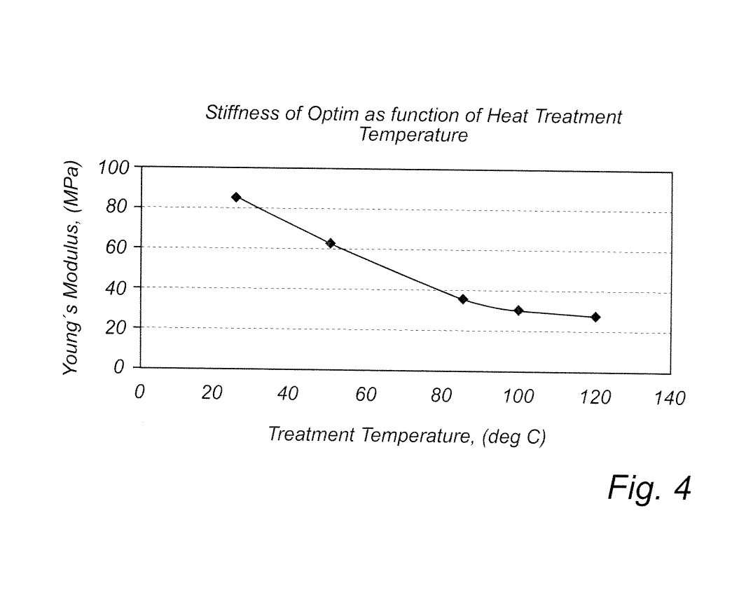

FIG. 4 shows the relationship between stiffness, indicated by Young's Modulus, and treatment temperature of a polymer sheet material according to an embodiment of the invention. The experiment was performed by first heat treating an Elast-Eon 2A material in a conventional oven. In FIG. 4, the name Optim is used which is a name of the Elast-Eon 2A material used at St. Jude Medical. Thereafter, the stiffness was then measured by a conventional apparatus for measuring tensile properties of stress versus strain. Lloyd Instruments LRX plus ExT with 10N load cell tested on tubing in a mandril clamp with 100 mm gauge length. The graph shows that a higher treatment temperature results in a lower stiffness of the polymer material or Elast-Eon 2A provide by Aor-Tech.

### Heat Treatment Process

As is understood by the skilled person in the art, the heat treating process according to the present invention may be performed in number of alternative ways. In an example method for manufacturing of an implantable polymer sheet material which is to be implanted into a patient according to the present invention, first a continuous sheet of a polymer material is provided. Thereafter, at least a proximal portion of the polymer sheet material is processed in at least a first heat process stage. Thereby, an inherent resistance to wear of the polymer sheet material is substantially maintained. Thereafter, a distal portion in at least a second heat process stage is processed. The polymer morphology of this polymer material is altered such that an inherent flexibility of said polymer sheet material is substantially increased.

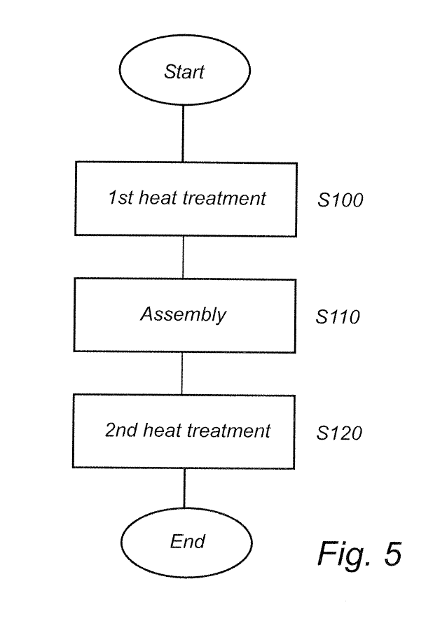

In FIG. 5, there is shown a schematic block diagram of a preferred process. First, at step S100, at least one polymer tube is placed in an oven having a temperature of about 85° C. The at least one polymer tube is annealed in a batch process over the full length of the tube to stabilize its dimensions for 4 hours. The temperature and/or time parameter may be varied within the interval of the present invention, i.e. a temperature interval from about 50° C. to about 100° C. during a period of about 30 minutes to about 5 hours, to achieve a desired stabilizing effect of the dimensions. However, a preferred first process steps is, as mentioned above, to subject the tube to a first heat treatment step S100 at a temperature of about 85° C. for about 4 hours. Thereafter, at step S110, a lead is assembled using a processed polymer tube as an outer tube. This is not described in detail since is conventional practice within the art. After assembly of a lead, the lead is heat treated in a second heat treatment step S120. Preferably, a heating mantle or other suitable localized controlled temperature heat source is used to treat the sections of the lead, preferably about 10-25 cm of the distal lead end, where a higher degree of flexibility is desired. Temperatures selected influence the modulus or stiffness of the material in a controlled fashion. However, the second heat treatment is preferably performed at a temperature of about 120° C. for about 30 minutes.

As is understood, the tube or polymer sheet material may be gradually heated to attain a mechanical property gradient, i.e. the flexibility at the distal end is gradually decreased towards the proximal end, or at least up to that part of the lead that is not be wear or abrasion resistant.

Also, as is understood by those skilled in the art, the method may also comprise the step of providing at least one electrical conductor adapted to be connected to at least one electrode and/or sensor of the lead. Also, the step of assembling the medical lead may be done in a number of alternative ways. For example, the assembly of the lead may be performed after completion of the first and second heat treatment steps, or may be performed before the heat treatment. However, a preferred embodiment is to assemble the medical lead after the first heat treatment. During the first heat treatment stage, relaxation of internal stresses can occur which may alter the dimensions of the polymeric component slightly. Thus, it is useful to treat the entire tube prior to the assembly in order to control tolerances of components of the medical lead and device. Moreover, the assembling may also be performed in a number of alternative ways. For example, the conductors may be positioned within the polymer sheet material after the first heat treatment step, followed by the second heat treatment step. However, this assembling may or may not include mounting the sensor and/or electrode to the lead and also the medical device or control unit may also be mounted in the same assembly step.

Although an exemplary embodiment of the present invention has been shown and described, it will be apparent to those having ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims.

1. (canceled)

2. The implantable medical lead according to claim 15, wherein the polymer is a semi-crystalline copolymer having at least a soft amorphous segment and at least a hard crystalline segment being at least partially crystallized.

3. The implantable medical lead according to claim 2, wherein at least a portion of the amorphous segment comprises at least one flexible polymeric material from a group containing silicone, polyethers, polyethylene oxide, polyolefins, polycarbonates, or a combination thereof, and wherein at least a portion of the crystalline segment comprises at least one crystallizable polymeric material from a group containing aromatic urea, aromatic or aliphatic urethane.

4. The implantable medical lead according to claim 15, wherein the at least said proximal portion of said polymer sheet material is heat treated at a temperature interval from about 50 to about 100° C. during a period of about 30 minutes to about 5 hours and said distal portion of said polymer sheet material is heat treated at a temperature at least about 10° C. above the temperature of the first heat process stage during a period of at least about 5 minutes.

5. The implantable medical lead according to claim 4, wherein said distal portion is heat treated at a temperature of about 120° C. during a period of about 30 minutes.

6. A method for manufacturing of an implantable polymer sheet material for implantation in a patient, wherein a distal portion of said polymer sheet material is adapted to be located in or at a heart of said patient and wherein a proximal portion of said polymer sheet material is connectable to an implantable medical device and arranged such that, when connected to said device, at least a part of said proximal is placed in close proximity to said medical device, said method comprising the steps of:
providing a continuous sheet of a polymer material;
processing at least said proximal portion of said polymer sheet material in at least a first heat process stage such that an inherent resistance to wear of said polymer sheet material is substantially maintained;
processing said distal portion in at least a second heat process stage in which a polymer morphology of said polymer material is altered such that an inherent flexibility of said polymer sheet material is substantially increased.

7. A method for manufacturing of an implantable medical lead for implantation in a patient, wherein a distal portion of said lead is adapted to be located in or at a heart of said patient and wherein a proximal portion of said lead is connectable to an implantable medical device and arranged such that, when connected to said device, at least a part of said proximal is placed in close proximity to said medical device, said method comprising the steps of:
providing at least one electrical conductor connected to at least one electrode and/or sensor of said lead;
providing a continuous sheet of a polymer material;
processing at least said proximal portion of said polymer sheet material in at least a first heat process stage such that an inherent resistance to wear of said polymer sheet material is substantially maintained;
processing said distal portion in at least a second heat process stage in which a polymer morphology of said polymer material is altered such that an inherent flexibility of said polymer sheet material is substantially increased; and
assembling said at least one conductor with said polymer sheet material.

8. The method according to claim 6, wherein the step of providing a continuous sheet of a polymer material comprises a step of providing a semi-crystalline copolymer having at least a soft amorphous segment and at least a hard crystalline segment being at least partially crystallized.

9. The method according to claim 6, wherein the step of providing a continuous sheet of a polymer material comprises a step of providing a semi-crystalline copolymer having at least a soft amorphous segment where at least a portion thereof comprises at least one flexible polymeric material from a group containing silicone, polyethers, polyethylene oxide, polyolefins, polycarbonates, or a combination thereof, and having at least a hard crystalline segment where at least a portion thereof comprises at least one crystallizable polymeric material from a group containing aromatic urea, aromatic or aliphatic urethane.

10. The method according to claim 6, wherein the first heat process stage comprises heating within a temperature interval from about 50 to about 100° C. during a period of about 30 minutes to about 5 hours, and wherein the second heat process stage comprises heating at a temperature at least 10° C. above the temperature of the first heat process stage during a period of at least 5 minutes.

11. The method according to claim 10, wherein the second heat process stage comprises heating at a temperature of about 120° C. during a period of about 30 minutes.

12. The method according to claim 6, comprising conducting the first and second heat process stages simultaneously by using a common oven that provides individual heat treatments to individual portions of the polymer sheet material.

13.-14. (canceled)

15. An implantable medical lead comprising:
a lead body adapted for in vivo implantation in a patient, said lead body being formed by a continuous sheet of polymer material, said polymer material having an inherent resistance to wear and an inherent flexibility;
at least one electrical conductor contained in said lead body in said continuous sheet of polymer material;
said lead body comprising a distal end adapted for location at least in proximity to the heart of the patient, and a proximal end configured for connection to an implantable medical device, and said continuous sheet of polymer material comprising a distal portion adjacent said distal end of said lead body and a proximal portion adjacent said proximal end of said lead body;
said proximal portion of said continuous sheet of polymer material being processed in a first heat processing stage that substantially maintains said inherent resistance to wear of said polymer material; and
said distal portion of said polymer heat material being processed in a second heat processing stage that alters a polymer morphology of said polymer material to substantially increase said inherent flexibility of said polymer material.

16. The method according to claim 7, wherein the step of providing a continuous sheet of a polymer material comprises a step of providing a semi-crystalline copolymer having at least a soft amorphous segment and at least a hard crystalline segment being at least partially crystallized.

17. The method according to claim 7, wherein the step of providing a continuous sheet of a polymer material comprises a step of providing a semi-crystalline copolymer having at least a soft amorphous segment where at least a portion thereof comprises at least one flexible polymeric material from a group containing silicone, polyethers, polyethylene oxide, polyolefins, polycarbonates, or a combination thereof, and having at least a hard crystalline segment where at least a portion thereof comprises at least one crystallizable polymeric material from a group containing aromatic urea, aromatic or aliphatic urethane.

18. The method according to claim 7, wherein the first heat process stage comprises heating within a temperature interval from about 50 to about 100° C. during a period of about 30 minutes to about 5 hours, and wherein the second heat process stage comprises heating at a temperature at least 10° C. above the temperature of the first heat process stage during a period of at least 5 minutes.

19. The method according to claim 18, wherein the second heat process stage comprises heating at a temperature of about 120° C. during a period of about 30 minutes.

20. The method according to claim 7, comprising conducting the first and second heat process stages simultaneously by using a common oven that provides individual heat treatments to individual portions of the polymer sheet material.
